(19) United States
(12) Patent Application Publication
(10) Pub. No.: US 2022/0323621 A1
(43) Pub. Date: Oct. 13, 2022

(54) METHOD OF STERILIZATION OF BIOLOGICS

(71) Applicant: Hyalo Technologies, LLC, Somerset, NJ (US)

(72) Inventor: Shalabh JAIN, Somerset, NJ (US)

(73) Assignee: Hyalo Technologies, LLC, Somerset, NJ (US)

(21) Appl. No.: 17/716,360

(22) Filed: Apr. 8, 2022

Related U.S. Application Data
(60) Provisional application No. 63/173,157, filed on Apr. 9, 2021.

(51) Int. Cl. A61L 2/00; A61L 2/20

(52) U.S. Cl. CPC A61L 2/0094; A61L 2/0023; A61L 2/206; A61L 2202/21; A61L 2101/44

(57) ABSTRACT

Methods of sterilizing biologics or biological components are disclosed wherein the biologic or biological component in solution or suspension form are formed using an annealing step during freeze drying so that a porous solid matrix which allows penetration of a sterilizing gas such as EtO to pass through. The annealing process decreases the particle size of lyophilized material as compared to other methods and provides a more uniform cake that is easy to reconstitute. In addition, the resulting lyophilized material made with the annealing step allows better penetration of the sterilizing gas for more effective and uniform sterilization of the material.

### CROSS-REFERENCE TO RELATED APPLICATIONS

This application claims the benefit of priority from U.S. Provisional Patent Application No. 63/173,157, filed Apr. 9, 2021, the disclosure of which is incorporated herein by reference.

### BACKGROUND OF THE INVENTION

The biotech and pharmaceutical industries often use biologics or biological components, such as vaccines, antibodies, gene therapies, plasmids, proteins, allergens, tissues, attenuated viruses, hormones, enzymes, blood, blood components or biosimilars for various diagnostic and therapeutic treatments. Any alteration in these structures can either decline or eliminate their function in vivo. For example, proteins can be damaged, due to change in pH, non-covalent bonds broken due to application of heat, interactions with the surrounding molecules or solvents leading to chemical changes in the molecular structure, thus rendering the protein unusable. These molecules are also susceptible to damage if the final formulations are stored in liquid form at refrigeration or low temperatures. Even storage in harsh conditions, extreme heat or frozen storage conditions can lead to degradation of the product more quickly and decrease the shelf life of the product.

Despite the vast benefits, biologics can also easily be contaminated with various biological contaminants ranging from viruses to bacteria. These contaminants not only may cause serious health issues when injected, but also have the ability to reduce the efficacy and destroy the materials that they contaminate. These products have to be either prepared by using pre-sterilized ingredients in aseptic conditions or have to be sterilized after preparation, which can be cumbersome, require special facilities and equipment or involve methods that can use heat, moisture, gamma radiation, or exposure to solvents. Therefore, biologics need special handling and storage conditions since they are susceptible to degradation by environmental conditions, such as temperature changes, light, shear and exposure to different sterilization methods or conditions.

### SUMMARY OF THE INVENTION

The methods of the invention provide improved sterilization of biologics and biological components. Some biological components such as antibodies, are water soluble and will make a clear solution in the matrix. Other biological components such as vaccines, can consist of microparticles and will make a suspension in the matrix. The matrix described in this invention works for both type of biological components due to its ability to uniformly suspend the biological components . The resultant products obtained by the processes described herein have improved stability and allow sterilization to be carried out on temperature sensitive biologics or the like without significant loss of activity.

The methods include a) forming an aqueous dispersion, containing a biologic or biological component, a viscosity inducing polymer, a stabilizer and optionally a wetting agent; b) reducing the temperature of the aqueous dispersion to a first freezing temperature for a first time period to form a frozen composition; c) increasing the temperature of the frozen composition to an annealing temperature for a second time period; d) decreasing the temperature of the frozen composition after the second time period to a second freezing temperature; e) lyophilizing the frozen composition to form a porous polymer matrix in which the biologic or biological component is substantially dispersed; and f) exposing the lyophilized porous polymer matrix containing the biologic or biological component substantially dispersed therein to a sterilizing gas under conditions to sufficient to substantially sterilize the lyophilized porous matrix containing the biologic or biological component.

Further aspects of the invention include preparing a solution or suspension of the sterilized biological component by reconstituting the porous matrix containing the biological component.

Some of the advantages gained by the processes of the present invention include—the annealing process decreases the particle size of lyophilized material as compared to other methods and provides a more uniform cake that is easy to reconstitute. In addition, the resulting lyophilized material made with the annealing step allows better penetration of the sterilizing gas for more effective and uniform sterilization of the material.

### BRIEF DESCRIPTION OF THE DRAWINGS

FIG. 1 is a comparative graph illustrating the amount of papain recovered after reconstitution of freeze dried matrices sterilized according to the invention vs. Control in accordance with Example 1.

FIG. 2 is a graph illustrating the amounts of antibody recovered after sterilization processes described in Example 2.

### DETAILED DESCRIPTION OF THE INVENTION

In accordance with a first aspect of the invention, there are methods of sterilizing biologics or biological materials. The methods generally include:
a) forming an aqueous dispersion containing a biologic or biological component, a viscosity inducing polymer, a stabilizer and optionally a wetting agent;
b) reducing the temperature of the aqueous dispersion to a first freezing temperature for a first time period to form a frozen composition;
c) increasing the temperature of the frozen composition to an annealing temperature for a second time period;
d) decreasing the temperature of the frozen composition after the second time period to a second freezing temperature;
e) lyophilizing the frozen composition to form a porous polymer matrix in which the biologic or biological component is substantially dispersed; and
f) exposing the lyophilized porous polymer matrix containing the biologic or biological component substantially dispersed therein to a sterilizing gas under conditions to sufficient to substantially sterilize the lyophilized porous matrix containing the biologic or biological component.

For purposes of the present invention, the terms “biologic” and “biological component” shall be understood to broadly include any substance having biological activity when administered to man or animal. In accordance therewith, biologics and biological components can include without limitation vaccines, antibodies, antibody fragments, gene therapies, plasmids, plasmid fragments, proteins, allergens, tissues, attenuated viruses, hormones, enzymes, blood, blood components and mixtures thereof.

Suitable viscosity inducing polymers include materials that are preferably hydrophilic polymers which are soluble or partially soluble in water and impart sufficient viscosity to the solution. Examples of such polymer include cellulose derivatives such as hydroxypropyl cellulose, hydroxypropyl methyl cellulose, carboxymethyl cellulose, and hydroxyethyl cellulose. Alternatives include alginic acid and its derivatives, carrageenan, and polyethylene glycol. These polymers may also be used as their salts, for example, carboxymethyl cellulose may be used as the sodium salt. One preferred polymer used in this invention is sodium carboxymethyl cellulose (referred to herein as Na CMC or CMC) such as those wherein the polymer has MW between50,000 to >80000 or 70,000 to over 250,000. Other viscosity inducing materials with similar properties may also be suitable. It is this viscosity inducing material or polymer which forms the matrix which supports the biological component after lyophilization of the dispersion containing the viscosity inducing polymer, stabilizer and optional wetting agent and biological component. Thus, the viscosity inducing polymer is used synonymously herein with the term “matrix forming polymer”. The amount of viscosity inducing polymer included in the aqueous suspension can range from about 0.1 to about 2.0% w/v.

The aqueous dispersion also includes a stabilizer. For example, other polymers or small molecules may be added to protect the biologic or biological components during the process of freezing and drying. Examples of small molecules include polyhydric sugars such as sucrose, mannitol, and trehalose. Glycine or other cryo-preservatives and lyo-preservatives known to persons skilled in art can be included as well if desired. In many embodiments, the preferred stabilizer is mannitol. The stabilizer will be included in the aqueous dispersion prior to lyophilization at a concentration range of 5-15% w/v.

The aqueous suspension optionally includes a wetting agent such as an ionic or non-ionic surfactant, which aids in forming a substantially uniform suspension of particles by improving wetting of particles by the solvent. These surfactants can be non-polymeric small molecules or polymeric in nature. Examples of some preferred polymeric surfactants are polyvinyl pyrrolidone (PVP) and polyvinyl alcohol (PVA). The amount of wetting agent, when included in the dispersion, can range from about 0.1 to about 0.3% w/v . In some alternative aspects the wetting agents include polyethylene glycol, Tween 20 and Tween 80. The wetting agent is preferably included as part of the aqueous dispersion and thus the subsequent protective matrix made therefrom when the sterilization is carried out at temperatures of greater than about 45° C. and more preferably when the sterilization is carried out at temperatures of about 50° C. or higher.

The pH of the aqueous solution prior to the addition of the biologic is usually between about 4.0 to about 8.0 and is preferably from about 6.0 to about 7.5. The water soluble viscosity inducing polymer preferably being NaCMC, the stabilizer preferably being mannitol; and the wetting agent preferably being polyvinylpyrrolidone (PVP).

It is to this aqueous dispersion that the biologic is added, thereby forming the dispersion, which is frozen, annealed, eventually lyophilized, sterilized and then reconstituted upon need.

The first part of the process includes forming an aqueous suspension containing the biologic or biological component. This is done using standard laboratory mixing techniques, combining the biologic, a viscosity inducing polymer a stabilizer and, optionally, wetting agent. Other ancillary ingredients can be included if desired as well. The aqueous dispersion preferably has a viscosity of from about 10 to about 2,000 centipoise and in alternative embodiments, the viscosity of the aqueous suspension is from about 10 to about 1,000 centipoise. The concentration of the biologic or biological component in the aqueous dispersion can be from about 0.1 to about 500 milligrams per milliliter and in some alternative embodiments, it will be from about 1 to about 100 milligrams per milliliter. The concentration may vary outside these ranges so long as the suspension is capable of undergoing the process under which the resultant matrix can be annealed and sterilized.

Once the aqueous suspension is formed, it is then frozen. Specifically, the temperature of the aqueous dispersion is reduced to a first freezing temperature for a first time period to form a frozen composition. The first freezing temperature is a temperature which is preferably about −40° C. or lower. While the first time period is at least about 1 hour and can in some alternative embodiments be in the range of from about 1 hour to about 10 hours. The amount of time that this frozen composition, sometimes referred to herein as a matrix, is held at the first freezing temperature can generally be regarded as the amount of time which is sufficient to ensure that the water in the aqueous suspension containing the biologic is converted to ice and then is separated from the highly concentrated solute molecules.

The next step of the process is referred to herein as the annealing step in which the temperature of the frozen composition is increased to a temperature which is higher than the first freezing temperature but still below the freezing temperature of water, referred to herein as the annealing temperature for a second time period. The annealing temperature is at least about 10° C. higher than the first freezing temperature and in many aspects of the invention, the temperature of the frozen composition is gradually raised until the temperature is from about −10° C. to about −1° C. The amount of time the frozen composition is maintained at the annealing temperature second time period is at least about 1 hour and in many aspects of the invention, it can be from about 1 hour to about 10 hours or longer if desired.

The annealing portion of the process is one in which the frozen composition taken from the first freezing temperature and then gradually warmed by from about 10° C. to about 40° C. and held at this annealing temperature for at least an hour and preferably several hours. For example, in many embodiments, the temperature is gradually raised from the first freezing temperature, e.g. −40° C. or lower to a higher, but still freezing temperature, e.g. about −15 or as high as about −1.0° C. and held there from about 1 hour to about 10 hours. For purposes of the present invention, it will be understood by those of ordinary skill that the annealing temperature is a temperature which lies below the melting point temperature of ice and above the glass transition temperature of the solute concentrate. While not wishing to be bound by theory, the modification in the freezing step of the lyophilization is believed to crystallize the matrix forming ingredients. The crystallization of the ingredients helps provide a framework or network to protect the actives in the matrix and formation of pores that help in the later, easy penetration of the sterilizing gas.

After the annealing step is completed, the temperature of the frozen composition is again lowered to a second freezing temperature and held for a third time period. The second freezing temperature can be the same as the first freezing temperature, i.e. about −40° C. or lower, but need not exactly match the first freezing temperature. Similarly, the third time period can be the same as the first time period, i.e. at least about an hour or more, but is not necessarily the same.

Once the second freezing temperature has been reached for a sufficient time which can be a few hours, for example, the frozen composition is lyophilized to form a porous polymer matrix in which the biologic or biological component is substantially dispersed. This step causes the frozen composition to be dried under high vacuum to allow all the solvent to evaporate from solid to vapor state and form a solid porous matrix.

During drying under high vacuum, the product temperature may be raised to higher than −40° C. to increase the drying speed if desired. This matrix stabilizes the active ingredients and allows them to be sterilized at or close to room temperature without involving any aseptic conditions if desired. This process step of lyophilization or freeze drying is well known to those of ordinary skill and for the sake of brevity is not expounded upon herein. The result of lyophilization is a porous cake which is essentially devoid of any solvent. This leaves the biologic within a solid porous cake comprising of a uniform suspension of the biological components in a solid matrix. This matrix is stable at room temperature and can be subject to sterilization by a variety of techniques, including gas sterilization.

In accordance with this sterilization step, the lyophilized porous polymer matrix containing the biologic or biological component substantially dispersed therein is subjected to a sterilizing gas under conditions to sufficient to substantially sterilize the lyophilized porous matrix containing the biologic or biological component. These conditions sufficient to substantially sterilize the lyophilized porous polymer matrix containing the biologic or biological component include in some aspects of the invention carrying out the sterilizing at a temperature of from about 20 to about 60° C., while in other aspects, the temperature is ≤ about 38° C. or from about 37 to about 55° C. In alternative embodiments, temperatures above 55° C. can be used.

The sterilizing gas is preferably ethylene oxide although alternatives can be used if desired. Other sterilization gases will be apparent to those of ordinary skill. For purposes of the present invention, “conditions sufficient to substantially sterilize” shall be understood to include those conditions, i.e. gas concentration, humidity, and time typically used while carrying out EtO or other suitable gas sterilization at the temperatures described herein.

In some optional aspects of the invention, a second or multiple annealing step(s) is/are carried out before lyophilization. In such aspects, after the lower freezing temperature has been reached and maintained for a desired period of time, the frozen composition is subjected to a second or further annealing step, which, like the first involves gradually raising the temperature of the frozen composition or matrix to a temperature below the freezing temperature of water before, it is lowered again to a temperature which allows lyophilization to occur. It will also be clear that while in most aspects of the invention, the gas sterilization can be preferably carried out at temperatures from about room temperature to about 50° C., the sterilization can also be done at temperatures outside this range, if desired without undue experimentation.

In sum, the methods include dispersing the active biologic ingredient(s) in an aqueous solution containing viscosity inducing polymer, a stabilizer and a wetting agent; freezing the resultant composition, annealing the frozen composition or matrix, followed by lowering the temperature of the frozen matrix, lyophilizing the annealed matrix to produce a solid-state porous matrix upon drying which can easily sterilized and then solubilized in aqueous solvents to yield a clear viscous solution. Preferably, the lyophilized porous matrix is exposed to a sterilizing gas at a temperature at or close to room temperature conditions or higher temperatures in other embodiments such as ≤38° C. or in the range of 37° C.-50° C., or higher if desired. See, for example Jain, S. U.S. Pat. No. 10,821,200, the contents of which are incorporated herein by reference.

In accordance with another aspect of the invention, there are provided methods of reconstituting the sterilized, lyophilized porous matrix containing the biologic components. These methods include contacting the sterilized, lyophilized porous matrix containing the biological component with an amount of a reconstitution solvent sufficient to substantially re-suspend the biological components within the matrix. The reconstitution solvent is preferably water or an aqueous-based liquid.

Advantages of the Matrix and Method of Sterilization
This method enables sterilization to be carried out at a low temperature, which ensures drug product stability and is ideal for highly sensitive materials or macromolecules, such biologics.
The method aids in the sterilization of temperature sensitive ingredients.
The method eliminates the need for aseptic processing and sterile rooms during production.
The process of sterilization can be carried out in bulk or in single dose vials.
The final solid form of the product allows for easy filling of the dose in final containers.
The final formulation can be easily reconstituted with an aqueous solvent.
Improved stability of the final product increases shelf life, which allows for long term storage.
The formulation can be stored longer and safer at room temperature.
The method reduces the weight and volume of the final product.

### EXAMPLES

The following examples serve to provide further appreciation of the invention but are not meant in any way to restrict the effective scope of the invention.

### Example 1

### Matrix Containing a Protein Molecule

The active ingredient Papain can be dispersed in the matrix with various ratios of the viscosity inducing agent, stabilizer and a wetting agent. An example of a ratio is below:

| Amount of Active | | | |
|---|---|---|---|
| Ingredient | Matrix Forming Ingredients | | |
| Papain | NaCMC | PVP | Mannitol |
| % w/v | % w/v | % w/v | % w/v |
| 0.02 | 1 | 0.2 | 5 |

The aqueous solution containing the above active ingredient, viscosity inducing polymer (NaCMC, 262.19 g/mol), wetting agent (PVP—molecular weight 111.14 g/mol) and stabilizer (mannitol—molecular weight, 182.172 g/mol) are lyophilized. In the freezing step, the temperature was lowered to −40 C for 2 hours, and then annealed by gradually raising the temperature of the frozen solute concentrate to −10 C and held at this temperature for 3 hours. The samples where then cooled to −40 C and held for 2 hours and then dried under a very high vacuum to allow all the solvent to evaporate and form a porous matrix which was then subjected to gas sterilization using ethylene oxide at ≤38° C. (low temperature) or 50° C. (high temperature).

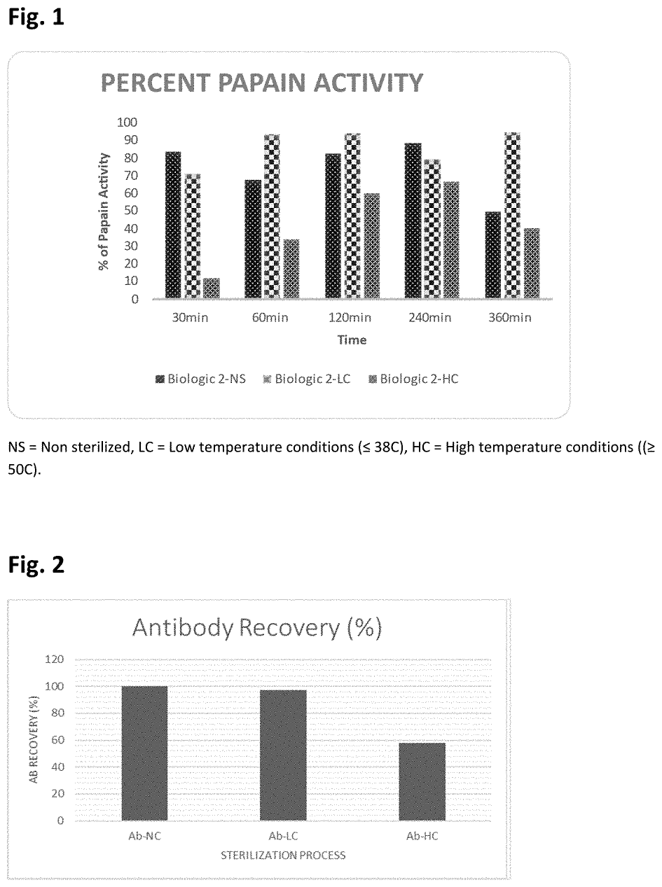

The sterilized and the non-sterilized matrix was then reconstituted with a known amount of the aqueous solvent and subjected to enzymatic activity assay to determine the loss of activity, if any during the process of sterilization at the times shown in FIG. 1. Enzymatic activity of papain was carried out by exposing an insoluble form of collagen to the reconstituted solution, followed by sampling at the indicated time points. The samples were assayed for the free protein concentration which results from digestion of collagen by the enzyme and constitutes a measure of the enzyme activity.

From the study we found that the amount of protein recovered when sterilized at low temperature conditions on average was higher up to 94%, when compared non-sterilized conditions up to 88% and at high temperature conditions up to 66%.

The above data shows that the active ingredient can retain its efficacy in the matrix. The data also shows that we can effectively sterilize the matrix or the dispersion containing the active through the process of ethylene oxide sterilization. The percentage recovery shows that the matrix helps preserve the effectiveness of the Papain molecule. The stability of the product is improved, as the formulations can now be stored at room temperature after sterilization.

### Example 2

### Matrix Containing a Biosimilar Antibody

| Amount of Active | | | |
|---|---|---|---|
| Ingredient | Matrix Forming Ingredients | | |
| Antibody | NaCMC | PVP | Mannitol |
| % w/v | % w/v | % w/v | % w/v |
| 0.1 | 0.5 | 0.2 | 10 |

The aqueous solution containing the biosimilar antibody infliximab-axxq , viscosity inducing polymer (NaCMC, 262.19 g/mol), wetting agent (PVP—molecular weight 111.14 g/mol) and stabilizer (mannitol—molecular weight, 182.172 g/mol) are lyophilized. In the freezing step, the temperature was lowered to −40° C. for 2 hours, and then annealed by gradually raising the temperature of the frozen solute concentrate to −10° C. and held at this temperature for 3 hours. The samples where then cooled to −40 C and held for 2 hours and then dried under a very high vacuum to allow all the solvent to evaporate and form a porous matrix which was then subjected to gas sterilization using ethylene oxide at 38° C. (low temperature) or 50° C. (high temperature).

The samples were stored at RT after sterilization. For HPLC analysis, the samples were reconstituted with purified water to obtain a final antibody concentration of 1 mg/ml. These samples were analyzed using reversed phase chromatography with UV detection. Relative areas of the antibody peaks were used to determine percent recovery.

As shown in FIG. 2, the biosimilar antibody shows significant recovery after being exposed to sterilization.

The data shows that relative to unsterilized sample, the sample sterilized at about 38° C. retains 97.3% of its content. When sterilized at 55° C., the recovery is about 58%.

### Example 3

The process of Example 2 is repeated except that the biologic active belonging to the category of therapeutic proteins such as insulin or growth hormone is used in place of the antibody.

### Example 4

The process of Example 2 is repeated except that a second annealing step is carried out before lyophilization. In this process, the matrix is first frozen at a temperature of −40° C. for 2 hours, followed by 2 cycles of annealing at higher temperatures. In the first annealing step, the temperature is slowly raised to a temperature of −10° C. and held there for 3 hours. The samples are then cooled to −40° C. and held for 2 hours. Next the samples are annealed a second time by being gradually raised to a temperature of −15° C. before being cooled to a temperature of −40° C. and then dried under a very high vacuum to allow all the solvent to evaporate and form a porous matrix which was then subjected to gas sterilization using ethylene oxide at 38° C. (low temperature) or 50° C. (high temperature) and compared to a non-sterilized reference standard where favorable results are found.

### CONCLUSION

The matrix containing biologics, such as vaccines, antibodies, gene therapies, plasmids, proteins, allergens, tissues, attenuated viruses, hormones, enzymes, blood, blood components and biosimilars can be effectively sterilized at a lower temperature to prevent product denaturation, maintain efficacy, and enhances stability. The final product can then be stored at room temperature to prevent the use of any specialized refrigerated or low temperature storage conditions. The matrix not only eliminates the need for specialized storage and transportation, but also eliminates the need for aseptic processing allowing for sterilization to occur at the end of the manufacturing process.

### REFERENCES

Manders C. D, Manders E. K (2010). Sterilization, stabilization and preservation of functional biologics, European Patent No. EP1511377A1, the contents of which are incorporated herein by reference.
Wang B, Pikal M. J (Oct. 3, 2012). Stabilization of Lyophilized Pharmaceuticals by Process Optimization: Challenges and Opportunities. American Pharmaceutical Review, https://www.americanpharmaceuticalreview.com/Featured-Articles/122325-Stabilization-of-Lyophilized-Pharmaceuticals-by-Process-Optimization-Challenges-and-Opportunities/ the contents of which are incorporated herein by reference.

What is claimed is:

1. A method of sterilizing biologics or biological components, comprising:
a. forming an aqueous dispersion containing a biologic or biological component, a viscosity inducing polymer, a stabilizer and optionally a wetting agent;
b. reducing the temperature of the aqueous dispersion to a first freezing temperature for a first time period to form a frozen composition;
c. increasing the temperature of the frozen composition to an annealing temperature for a second time period;
d. decreasing the temperature of the frozen composition after the second time period to a second freezing temperature;
e. lyophilizing the frozen composition to form a porous polymer matrix in which the biologic or biological component is substantially dispersed;
f. exposing the lyophilized porous polymer matrix containing the biologic or biological component substantially dispersed therein to a sterilizing gas under conditions to sufficient to substantially sterilize the lyophilized porous matrix containing the biologic or biological component.

2. The method of claim 1, wherein the biologics or biological components are selected from the group consisting of vaccines, antibodies, antibody fragments, gene therapies, plasmids, plasmid fragments , proteins, allergens, tissues, attenuated viruses, hormones, enzymes, blood, blood components and mixtures thereof.

3. The method of claim 1, wherein the first freezing temperature is about −40° C. or lower.

4. The method of claim 1, wherein the first time period is at least about 1 hour.

5. The method of claim 4, wherein the first time period is from about 1 hour to about 10 hours.

6. The method of claim 1, wherein the annealing temperature is at least about 10° C. higher than the first freezing temperature.

7. The method of claim 6, wherein the annealing temperature is from about −10° C. to about −1° C.

8. The method of claim 1, wherein the second time period is at least about 1 hour.

9. The method of claim 8, wherein the first time period is from about 1 hour to about 10 hours.

10. The method of claim 1, wherein the second freezing temperature is about −40° C. or lower.

11. The method of claim 1, wherein the conditions sufficient to substantially sterilize the lyophilized porous polymer matrix containing the biologic or biological component include carrying out the sterilizing at a temperature of from about 20 to about 60° C.

12. The method of claim 11, wherein the temperature is ≤ about 38° C.

13. The method of claim 11, wherein the temperature is from about 37 to about 55° C.

14. The method of claim 1, wherein the sterilizing gas is ethylene oxide.

15. The method of claim 1, wherein the viscosity inducing polymer is a water soluble polymer or a cellulose derivative.

16. The method of claim 15, wherein the cellulose derivative is carboxy methylcellulose or the sodium salt of carboxy methylcellulose.

17. The method of claim 1 wherein the wetting agent is selected from the group consisting of polymeric and non-polymeric surfactants.

18. The method of claim 1 wherein the concentration of the biologic or biological component in the aqueous dispersion of step a) is from about 0.1 to about 500 milligrams per milliliter.

19. The method of claim 1, wherein the aqueous suspension of step a) comprises:
i) from about 0.1 to about 2.0% w/v water soluble, viscosity inducing polymer;
ii) from about 5 to about 15% w/v stabilizer; and
iii) from about 0.1 to about 5% w/v wetting agent.

20. The method of claim 1, further comprising suspending the sterilized lyophilized porous matrix containing the biologic or biological component in a solvent.

21. The method of claim 1, wherein a second annealing step is carried out after step d) followed by a third freezing step before step e).
